(19) United States
(12) Patent Application Publication
(10) Pub. No.: US 2005/0175558 A1
(43) Pub. Date: Aug. 11, 2005

(54) Method and process for detecting a nail surface

(76) Inventors: Scott L Nielson, Layton, UT (US); Craig P Gifford, West Jordan, UT (US)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(21) Appl. No.: 10/708,065

(22) Filed: Feb. 6, 2004

(51) Int. Cl. A61K 7/04; G06K 9/00

(52) U.S. Cl. 424/61; 382/127

(57) ABSTRACT

The invention is a method and process of facilitating detection of a nail surface by digitizing devices. The detection method and process is facilitated by applying a coating composition or a stencil to the nail surface. The method and process utilizes a coating composition which is applied to the nail surface. The coating composition is derived from any substance that can be applied to a nail surface, which may include opaque and/or matte type paints or stencils which digitizing devices would be benefited to utilize in distinguishing the nail surface from surrounding tissue. The coating composition may be applied permanently or temporarily. The coating composition may be soluble or non-soluble. The simple application method and process results in a wide scope for application as, for example, to a fingernail or toenail.

### BACKGROUND OF INVENTION

While working with natural fingernails for several years all of the digitizing systems, which were encountered, proved to be impractical in distinguishing a nail surface from its surrounding tissue. In most cases, the digitizer would capture all of the object data but required manual operator intervention in distinguishing between the nail surface and its surrounding tissue. The obvious defect with existing technologies is that the digital data acquired is subject to human error and requires tremendous amounts of time and skill to successfully distinguish the nail surface from its surrounding tissue. The objective of this invention is to create a method and process to separate the nail surface from the surrounding tissue. Further objectives include the ability to automatically edit the nail surface data from the surrounding tissue data with software. By using the aforementioned techniques, this invention can now accomplish the final result with relative ease.

### SUMMARY OF INVENTION

This is a method and process to distinguish a nail surface from its surrounding tissue by using a coating composition. This invention makes it easier and more accurate to acquire the digital data representing a nail surface. Prior to this invention most objects are scanned by themselves, such as a bolt or screw, and there is no easy method to distinguish the details of the scan data to separate the various objects contained in the data acquisition file. Today it is very easy to apply a substrate to most objects and acquire the information related to the object. However, when dealing with more than one object at a time in the same data acquisition file, such as a nail surface and its surrounding tissue, the existing approach becomes impractical and laborious. The method and process devised by this invention makes the problem of distinguishing objects within the same data acquired easy and trivial and hence a simple solution to the problems at hand.

### BRIEF DESCRIPTION OF DRAWINGS

FIG. 1 is a diagram of the preparation step embodied in the invention.

FIG. 2 is a diagram of the application of the coating composition step embodied in the invention.

FIG. 3 is a diagram of the step comprising the digitizing of the nail surface area embodied in the invention.

### DETAILED DESCRIPTION

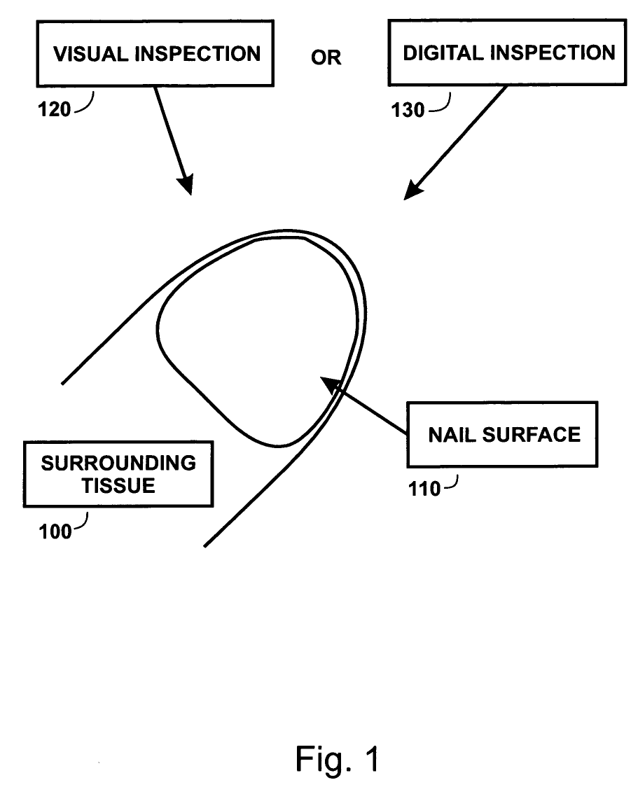

FIG. 1 is an illustration of a preferred embodiment of the invention with regards to the step of preparation of the object to be digitized. In this example, the finger is illustrated and the overall step involves the step of visually inspecting 120 the object including the surrounding tissue 100 and the nail surface 110. Visual inspection 120 involves the method and process of ascertaining any surrounding tissue 100 overages upon the nail surface 110 and the best way to remove any surrounding tissue, if necessary, to expose the appropriate nailsurface 110 required for successful digitizing. At this stage of the invention the visual inspection 120 is used to determine the best method and process for continuing to the next stage of the invention to digitizing the nail surface 110 object. Visual inspection 120 when done correctly will have removed all surrounding tissue 100 in relationship to the nail surface 110 exposing the nail and its periphery from tip to cuticle. During the visual inspection 120 it is determined if a manicure/pedicure or other finish work needs to be performed in order to effectively prepare the nail surface 110 for digitizing.

It is anticipated that a digital inspection 130 may occur to compare the object against other similar objects to determine if the object needs additional finish work before proceeding to the next stage of the invention. It is estimated that digital inspection 130 may take the forms of photographic images, laser imaging, refracted light imaging and mechanical measurements; other technologies may be utilized to digitally capture the object and do comparison and analysis work. It is further likely that no additional finish work need be performed to prepare the object for digitizing, as a result of the preparation step, in which case the preparation step would be concluded.

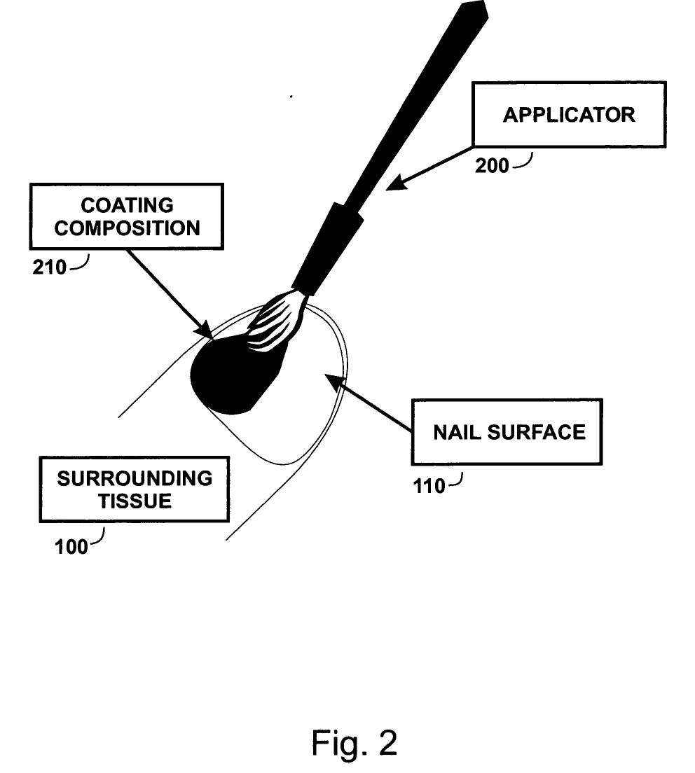

As shown in FIG. 2, the preparation step has been successfully concluded and the next step projected is the application of the coating composition 210. The coating composition 210 may be composed of any type of opaque, non-transparent, impenetrable, obscured, glossy, luminescence or semi-gloss substance, paint, veneer, covering, layer, dye or coating. Additional forms of coverings may include stencils or cutouts, which are designed to fit the nail surface 110 or the surrounding tissue 100 and distinguish the nail surface 110 from the surrounding tissue 100. In its preferred embodiment, the invention would be some type of non-toxic opaque and matte painting composition which can be applied and then removed after digitizing to the nail surface 110. The application of the coating composition 210 may be done uniformly and evenly with a thickness of the coating composition once this step is completed not to exceed 5 millimeters (five millimeters).

It is expected that the coating composition 210 is applied with an applicator 200, which reasonably permits the successful application of the coating composition 210 in an even and uniform method and process. The applicator 200 may take many forms, those anticipated for this invention include that of a brush, air brush, spray, dipping, cutouts, and stencils.

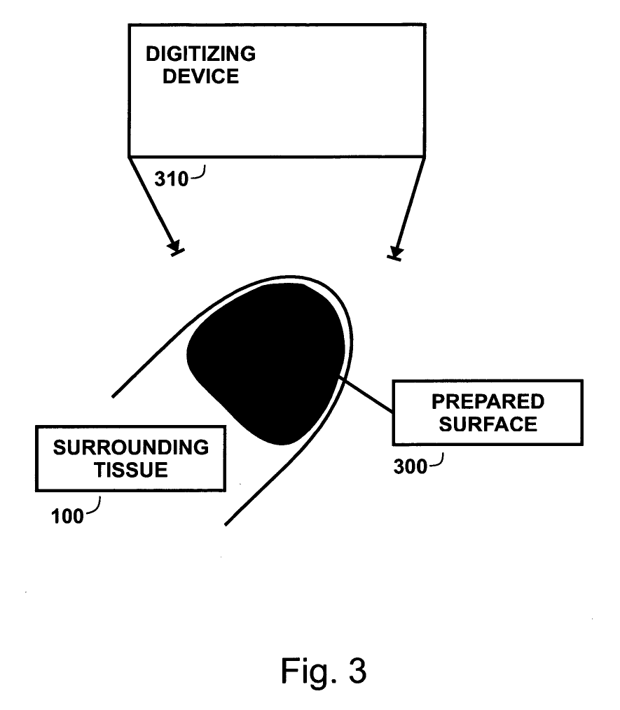

Finally in FIG. 3 the step of digitizing the object occurs. At this point, the nail surface 110 has been properly prepared and coated and becomes a prepared surface 300. A digitizing device 310 may be employed at this point to gather numerical and/or color and/or spatial data of the object. It is anticipated that the digitizing device 310 would render the above data to a computer in computer recognizable formatting including, but not limited too: numbers, scientific notation, RGB color data, CMYK color data, gray scale color data, XYZ point data, vertices, point clouds, or any type of geometrical shapes and lines.

The inspection and application of the coating composition steps make the digitizing of the object particularly easy and simple. By successfully completing the previous steps, virtually any digitizer would be capable of separating the nail surface object information into any computer recognizable format. The entire objective of the preferred embodiments of the invention has been to create an easy method and process to separate object information, specifically a nail surface 110 from its surrounding tissue 100. The application of this invention is extensive and plentiful, as with this invention it will become trivial to digitize an object and quickly and easily distinguish multiple objects within the same digital information gathered. Because of the advantages inherent in this invention it is anticipated that many variants of this invention are possible, which should be included within the preferred embodiments and descriptions of this invention.

1. A method for facilitating the detection of nail surfaces utilizing a coating composition, comprising:
preparation of the nail surface; and
application of the coating composition to the nail surface area or the surrounding tissue; and
digitizing the nail surface area.

2. The method of claim 1, wherein said step of preparation of the nail surface further comprises an inspection of each nail surface to determine the correct procedure to finalize preparation of the nail surface for the detection facilitating coating composition.

3. The method of claim 2, wherein the inspection further comprises:
a visual inspection of the nail surface; and/or
a computerized inspection of the nail surface by digitizing the nail surface.

4. The method of claim 2, wherein said step of determining the correct procedure to finalize preparation of the nail surface further comprises:
a manicure for fingernail surfaces; or
a pedicure for toenail surfaces.
However if the inspection method determines that the nail surface area is correctly prepared for the coating composition, this step may be omitted.

5. The method of claim 1, wherein said step of application of the coating composition to the nail surface area or the surrounding tissue may occur continuously or discontinuously and further comprises:
application of the coating composition by brushing the coating composition on the nail surface area or the surrounding tissue; and/or
application of the coating composition by spraying the coating composition on the nail surface area or the surrounding tissue; and/or
application of the coating composition by air brushing the coating composition on the nail surface area or the surrounding tissue; and/or
application of the coating composition by dipping the nail surface area into the coating composition; and/or
application of the coating composition by mechanical application of the coating composition on the nail surface area or the surrounding tissue; and/or
application of the coating composition by use of cutouts; and/or
application of the coating composition by the use of stencils.

6. The method of claim 1, wherein the said step of application of the coating composition further comprises:
application of the coating composition being done uniformly and evenly to cover the exposed nail surface area or the surrounding tissue; and
application of the coating composition is done to a thickness not to exceed five (5) millimeters.

7. The method of claim 1, wherein the coating composition further comprises: any type of non-transparent, impenetrable, obscured or opaque substance, covering, veneer, layer, stain, dye, tincture, coating or paint of any color; and/or
any type of matte, lusterless, dull or non-shiny substance, covering, veneer, layer, stain, dye, tincture, coating or paint of any color; and/or
any type of glossy, shiny or luminescence covering, veneer, layer, stain, dye, tincture, coating or paint of any color, and/or
any type of cutout that is designed to fit the nail surface area or the surrounding tissue which distinguishes the nail surface from the surrounding tissue; or
any type of stencil that is designed to fit the nail surface area or the surrounding tissue which distinguishes the nail surface from the surrounding tissue.

8. The method of claim 1, wherein the step of digitizing further comprises:
utilizing a computer device to capture numerical and/or color and/or spatial data of a nail surface and/or the surrounding tissue; or
utilizing a laser device to capture numerical and/or color and/or spatial data of a nail surface and/or the surrounding tissue; or
utilizing a mechanical device to capture numerical and/or color and/or spatial data of a nail surface and/or the surrounding tissue; or
utilizing reflected light or an optic device to capture numerical and/or color and/or spatial data of a nail surface and/or the surrounding tissue.
Where, numerical data includes any representation of the nail surface by numbers or scientific notation; and
color data includes any representation of the nail surface by common computer color recognition methods including RGB (Red, Blue, Green) and/or CMYK (Cyan, Magenta, Yellow, Black) and/or gray scale; and
spatial data includes any representation of the nail surface by amplitude, breadth, width, length or expanse; further comprising any geometrical coordinate representation similar to an XYZ coordinate system where the data may reflect points and/or vectors and/or vertices to create any type of geometrical lines and/or shape(s).

9. A process for facilitating the detection of nail surfaces utilizing a coating composition, comprising:
preparation of the nail surface; and
application of the coating composition to the nail surface area or the surrounding tissue; and
digitizing the nail surface area.

10. The process of claim 9, wherein said step of preparation of the nail surface further comprises an inspection of each nail surface to determine the correct procedure to finalize preparation of the nail surface for the detection facilitating coating composition.

11. The process of claim 10, wherein the inspection further comprises:
a visual inspection of the nail surface; and/or
a computerized inspection of the nail surface by digitizing the nail surface.

12. The process of claim 10, wherein said step of determining the correct procedure to finalize preparation of the nail surface further comprises:
a manicure for fingernail surfaces; or
a pedicure for toenail surfaces.
However if the inspection method determines that the nail surface area is correctly prepared for the coating composition, this step may be omitted.

13. The process of claim 9, wherein said step of application of the coating composition to the nail surface area or the surrounding tissue may occur continuously or discontinuously and further comprises:
application of the coating composition by brushing the coating composition on the nail surface area or the surrounding tissue; and/or
application of the coating composition by spraying the coating composition on the nail surface area or the surrounding tissue; and/or
application of the coating composition by air brushing the coating composition on the nail surface area or the surrounding tissue; and/or
application of the coating composition by dipping the nail surface area into the coating composition; and/or
application of the coating composition by mechanical application of the coating composition on the nail surface area or the surrounding tissue; and/or
application of the coating composition by use of cutouts; and/or
application of the coating composition by the use of stencils.

14. The process of claim 9, wherein the said step of application of the coating composition further comprises:
application of the coating composition being done uniformly and evenly to cover the exposed nail surface area or the surrounding tissue; and
application of the coating composition is done to a thickness not to exceed five (5) millimeters.

15. The process of claim 9, wherein the coating composition further comprises:
any type of non-transparent, impenetrable, obscured or opaque substance, covering, veneer, layer, stain, dye, tincture, coating or paint of any color; and/or
any type of matte, lusterless, dull or non-shiny substance, covering, veneer, layer, stain, dye, tincture, coating or paint of any color; and/or
any type of glossy, shiny or luminescence covering, veneer, layer, stain, dye, tincture, coating or paint of any color, and/or
any type of cutout that is designed to fit the nail surface area or the surrounding tissue which distinguishes the nail surface from the surrounding tissue; or
any type of stencil that is designed to fit the nail surface area or the surrounding tissue which distinguishes the nail surface from the surrounding tissue.

16. The process of claim 9, wherein the step of digitizing further comprises:
utilizing a computer device to capture numerical and/or color and/or spatial data of a nail surface and/or the surrounding tissue; or
utilizing a laser device to capture numerical and/or color and/or spatial data of a nail surface and/or the surrounding tissue; or
utilizing a mechanical device to capture numerical and/or color and/or spatial data of a nail surface and/or the surrounding tissue; or
utilizing reflected light or an optic device to capture numerical and/or color and/or spatial data of a nail surface and/or the surrounding tissue.
Where, numerical data includes any representation of the nail surface by numbers or scientific notation; and color data includes any representation of the nail surface by common computer color recognition methods including RGB (Red, Blue, Green) and/or CMYK (Cyan, Magenta, Yellow, Black) and/or gray scale; and
spatial data includes any representation of the nail surface by amplitude, breadth, width, length or expanse; further comprising any geometrical coordinate representation similar to an XYZ coordinate system where the data may reflect points and/or vectors and/or vertices to create any type of geometrical lines and/or shape(s).
